(19) United States
(12) Patent Application Publication
(10) Pub. No.: US 2019/0351213 A1
(43) Pub. Date: Nov. 21, 2019

(54) PATIENT LINE DISLODGEMENT DETECTION DEVICE AND METHOD

(71) Applicant: Toltec Ventures LLC, Monument, CO (US)

(72) Inventor: Byron William Larson, Monument, CO (US)

(21) Appl. No.: 16/527,168

(22) Filed: Jul. 31, 2019

Related U.S. Application Data
(63) Continuation-in-part of application No. 15/150,873, filed on May 10, 2016, now Pat. No. 10,398,857.
(60) Provisional application No. 62/194,219, filed on Jul. 18, 2015.

(51) Int. Cl. A61M 39/28; A61M 5/168; A61M 5/50

(52) U.S. Cl. CPC A61M 39/281; A61M 2205/15; A61M 5/5086; A61M 5/16854

(57) ABSTRACT

An example patient line dislodgement detection device includes a body having a plurality of openings. The plurality of openings are each configured to receive a tubing line therethrough. The device is configured such that a force applied to a tubing line causes the body to restrict fluid flow through the tubing line. During use, the tubing line is received through the body of the dislodgement device. Fluid flow is restricted by the dislodgement device through the tubing line in response to application of a force on the tubing line. A dislodgement condition can be detected based on restricting the fluid flow through the tubing line.

### CROSS REFERENCE TO RELATED APPLICATIONS

This application is a continuation-in-part (CIP) of U.S. patent application Ser. No. 15/150,873 filed May 10, 2016, which claims the priority benefit of U.S. Provisional Patent Application No. 62/194,219 filed Jul. 18, 2015 for “Needle Dislodgement Detection System and Method,” each hereby incorporated by reference in its entirety as though fully set forth herein.

### BACKGROUND

Intravenous needles are used patients for fluid transfer, such as access to blood for testing (withdrawing blood), drug infusion, fluid nutrition infusion, and saline infusion, to name only a few examples. Other uses of intravenous needles include, but are not limited to, dialysis, other extracorporeal blood processing therapies, and blood transfusions. During use, intravenous needles and/or needle lines are typically secured to the patient via tape or other method. However, if a force is applied to the needle line, such as by the user pulling their arm while the needle line is caught, the needle may become dislodged. Therefore, detection of a dislodged needle is important for patient safety (e.g., to prevent a patient from bleeding to death).

### BRIEF DESCRIPTION OF THE DRAWINGS

FIG. 1 shows an example patient line dislodgement detection device.

FIG. 2 is a close-up view of the example patient line dislodgement detection device of FIG. 1.

FIG. 3 shows an example patient line dislodgement detection device in an unrestricted configuration.

FIG. 4A shows an example patient line dislodgement detection device in a restricted configuration illustrating a dislodgement condition.

FIG. 4B is a close-up view of the example patient line dislodgement detection device of FIG. 4A.

FIG. 5 shows another example patient line dislodgement detection device in a restricted configuration illustrating a dislodgement condition.

FIGS. 6A-6B show another example patient line dislodgement detection device.

FIGS. 7A-7C show another example patient line dislodgement detection device.

FIG. 8 shows another example patient line dislodgement detection device.

### DETAILED DESCRIPTION

A patient line dislodgement detection device and method is disclosed. An example patient line dislodgement detection device includes a body having an entrance and an exit. The entrance and the exit are each configured to receive a tubing line therethrough. Applying a force to a tubing line causes the body to restrict fluid flow through the tubing line, thereby indicating a potential dislodgement condition before the needle is dislodged, which can be implemented to prevent dislodgement.

An example patient line dislodgement detection method includes receiving a tubing line through a dislodgement device. The method also includes restricting fluid flow by the dislodgement device through the tubing line in response to application of a force on the tubing line. The method also includes detecting a dislodgement condition based on restricting the fluid flow through the tubing line.

It takes a certain amount of force to remove a taped needle from a patient's access site. The amount of force the device disclosed herein to restrict the needle line will be less than the force to pull the needle out of the access. This provides a level of engineering predictability that current clinical practice does not have.

In an example, the system and method operates under the force that a patient may apply to a needle line when moving, to cause a restricted flow of fluid to the patient. The restriction may be partial or complete. That is, fluid flow may be partially or completely (i.e., to the point of stopping fluid flow entirely). The restriction, in turn, enables a sensor (e.g., flow and/or pressure sensor(s)) to detect a condition caused by the restriction (e.g., change in flow and/or pressure) prior to the needle becoming dislodged. The condition (e.g., partial and/or complete restriction) may be detected and responded to appropriately.

The restriction may be detected by any suitable means, and is not limited to flow and/or pressure sensor(s). For example, the restriction may be detected by measuring a change in geometric, optical, capacitive, resistive, and/or other property of a component of the needle dislodgement system and/or the fluid caused by a restriction due to the applied force.

In an example, the system and method may implement the applied force to trigger a secondary mechanism which may be utilized for detection of the condition or stoppage of the flow. By way of illustration, a secondary mechanism may include clamping the line so as to reduce or eliminate blood loss from the patient.

Before continuing, it is noted that as used herein, the terms “includes” and “including” mean, but is not limited to, “includes” or “including” and “includes at least” or “including at least.” The term “based on” means “based on” and “based at least in part on.”

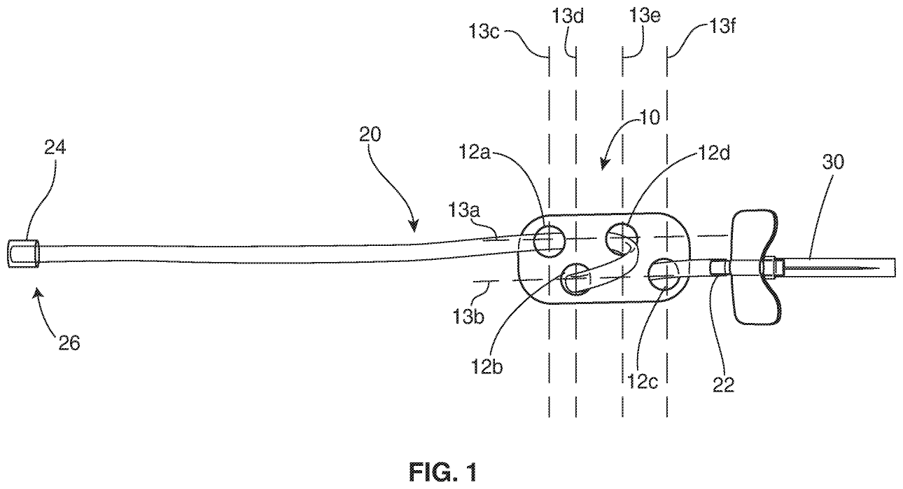

FIG. 1 shows an example patient line dislodgement detection device. In this example, the device 10 includes a body having openings 12a-d. The openings 12a-d are configured to receive a tubing line 20 for a patient. In an example, the tubing line 20 may be connected to a needle 30 (shown encased in a cover or shield) on one end 22 of the tubing line 20 and a fluid supply connection 24 on the other end 26 of the tubing line 20. By way of non-limiting illustration, the needle can be an intravenous needle. However, other needles for applying or retrieving other types of fluids to a patient may also be used.

It is noted that the device 10 is illustrative of an example configuration. The device 10 may have any suitable configuration, and is not limited to the configuration shown. Other configurations will be readily apparent to those having ordinary skill in the art after becoming familiar with the teachings herein.

The device 10 may be used with any patient line, such as lines to infuse fluid to and/or retrieve fluid from a human or veterinary patient. The device can similarly be applied to fluid connections to catheters for infusing or extracting fluids. In an example, a needle is attached to tubing line to extract or infuse fluids. These fluid lines are often attached to pumps, fluid bags, tees, and/or other connectors. Example fluids are blood, medications, saline, irrigation fluids, air (including suction), gases other than air, and nutritional products.

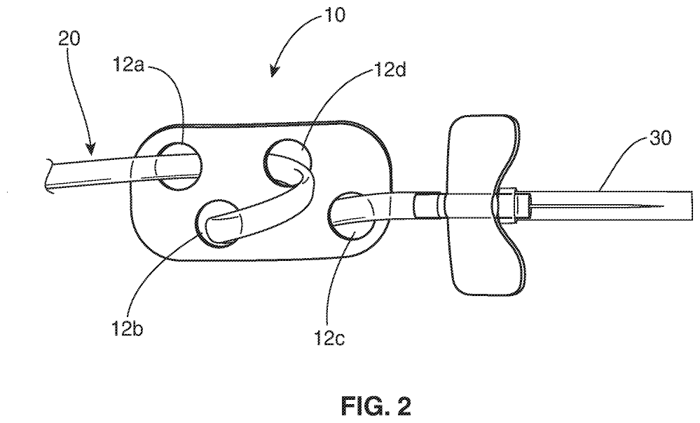

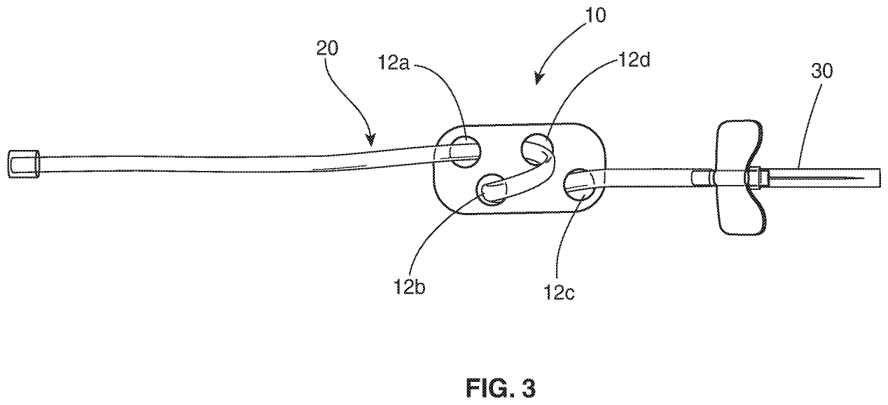

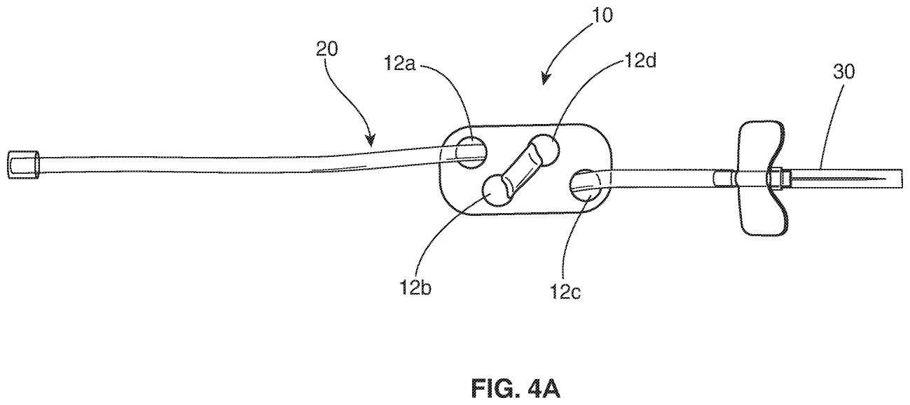

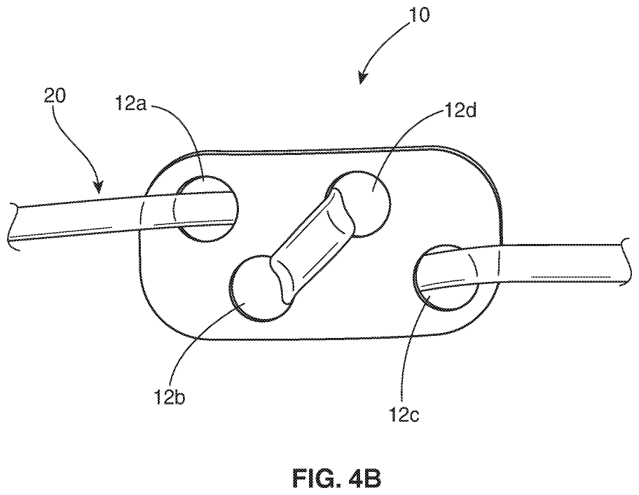

FIG. 2 is a close-up view of the example patient line dislodgement detection device 10 of FIG. 1. The patient line dislodgement detection device 10 is shown as it may include a body having an entrance and an exit. In an example, the entrance and the exit is formed by the openings 12a-d configured to receive a tubing line therethrough. Applying a force to the tubing line 20 (e.g., by pulling) causes the body to tighten within the openings (see, e.g., the illustrations of FIGS. 3 and 4 described below) and restrict fluid flow through the tubing line 20, thereby indicating a dislodgement condition.

In an example, the body is generally rectangular shaped, although other shapes, sizes, and configurations may also be provided. The body has a first opening 12a forming the entrance and a second opening 12c forming the exit. A third opening 12d and a fourth opening 12b reverse the direction of the tubing 20.

In an example, the first opening 12a and the third opening 12d are formed in the body along a first threading line 13a. The second opening 12c and fourth opening 12b are formed in the body along a second threading line 13b. The first threading line 13a may be spaced apart from the second threading line 13b. The first threading line 13a may be substantially parallel to the second threading line 13b.

The first opening 12a may be offset from the fourth opening 12b, as shown by lines 13c and 13d in FIG. 2. In addition, the second opening 12c may be offset from the third opening 12d, also as shown by lines 13e and 13f in FIG. 2. However, this configuration is not required in all examples.

In an example, the tubing line 20 is threaded over a first (e.g., top) side of the body and through the first opening 12a to a second (e.g., bottom) side of the body. The tubing line 20 is further threaded from the second side of the body through the third opening 12d to the second side of the body. The tubing line 20 is further threaded from the first side of the body through the fourth opening 12b to the second side of the body. The tubing line 20 is further threaded from the second side of the body through the second opening 12c to the first side of the body.

In an example use case, the line 20 may be pulled, such as may happen when the patient moves and dislodges the needle from a blood vessel. When the line 20 is pulled through the openings 12a-d, flow is at least partially restricted, if not fully blocked through the line 20. It is this restriction which can be detected using any suitable sensor or other detection device, such as but not limited to pressure and/or flow sensor(s).

As such, the device 10 enables enhanced detection of needle dislodgement. A dislodged needle can result in inappropriate fluid delivery (for example, inadequate drug delivery), fluid loss (for example, blood loss resulting in patient death), or inappropriate fluid extraction (for example, extraction of ambient air instead of a patient fluid).

FIG. 3 shows an example patient line dislodgement detection device in an unrestricted configuration. FIG. 4A shows an example patient line dislodgement detection device in a restricted configuration illustrating a dislodgement condition. FIG. 4B is a close-up view of the example patient line dislodgement detection device of FIG. 4A.

The patient line dislodgement detection method is based at least in part on the force on a needle line that might dislodge a needle or otherwise restrict the fluid flow. An example force may be a patient that inadvertently tugs on the fluid delivery line. The force is converted by the device to a means of restricting the needle line. The means may be a restricted line, a completely occluded line, or a valve that partially occludes after application of the aforementioned force. Other means are also contemplated. This fluid restriction provides needle dislodgement detection via a secondary sensing method. For example, pressure monitoring of the fluid infusion line, flow rate monitoring in the fluid infusion line, and/or other sensing that measures geometric changes due to the applied force.

An example patient line dislodgement detection method includes receiving a tubing line through a dislodgement device. The example method also includes restricting fluid flow by the dislodgement device through the tubing line in response to application of a force on the tubing line. The example method also includes detecting a dislodgement condition based on restricting the fluid flow through the tubing line. The example method also includes the dislodgement device restricting fluid flow in a needle line connected to a needle, catheter, or patient fluid connection.

In an example, the force at least partially restricts fluid flow in the tubing line. In another example, the force directly occludes the tubing line to completely stop fluid flow through the tubing line. Accordingly, the method includes measuring a physical change caused by partially or fully restricting the fluid flow. For example, measuring may include at least one of pressure sensing, flow rate sensing, and other restriction sensing caused by a strain on the tubing line 20. Measuring the physical change of the tubing line 20, the body, and/or the fluid in the tubing line 20 may include at least one of geometric, optical, capacitive, or resistive physical properties.

It is noted that at least partially (or fully) restricting the fluid flow may be by the body itself, and/or by a secondary device. Example secondary devices may include, but are not limited to a line clamp, a needle protection sheath, a wing, and an integral component of the tubing line.

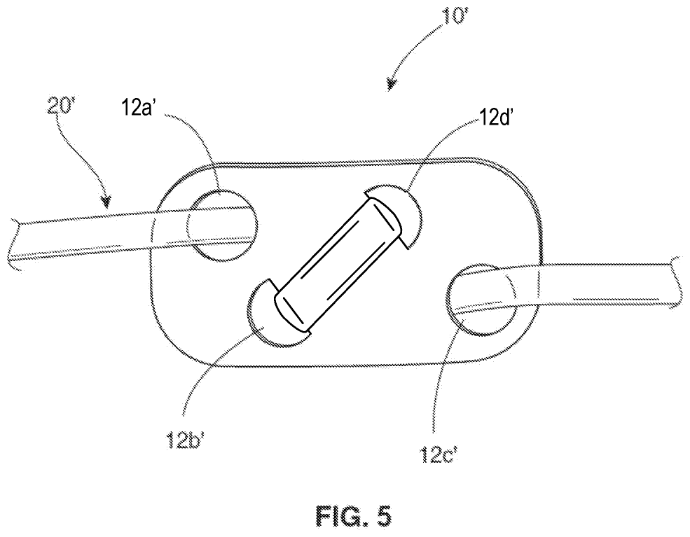

FIG. 5 shows another example patient line dislodgement detection device 10′ in a restricted configuration illustrating a dislodgement condition. In this example, prime reference numbers designate similar components already described above, and therefore are not described again here. In this example, at least two of the openings 12a′-12d′ each have at least one straight edge. For example, in the illustration shown, the openings 12b′ and 12d′ are substantially D-shaped. The straight edges may be aligned substantially parallel to one another. This configuration provides a flat surface to better occlude the tubing line 20′ when the force is applied to the tubing line 20′.

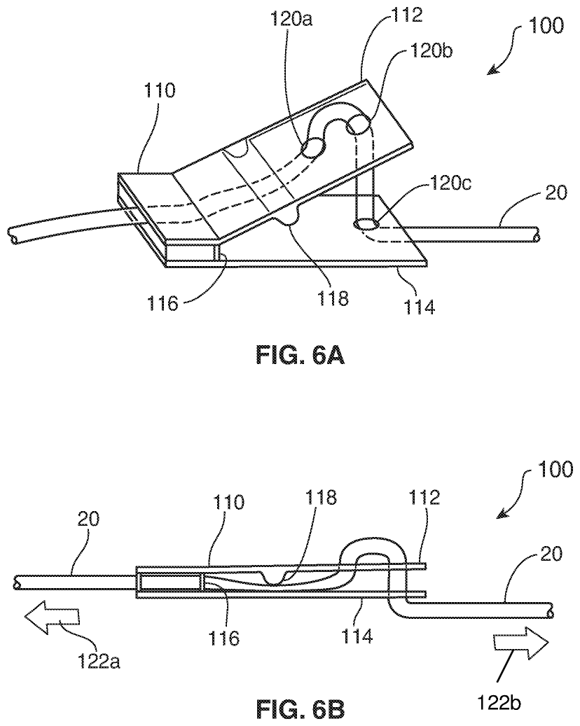

FIGS. 6A-6B show another example patient line dislodgement detection device 100. The example patient line dislodgement detection device 100 includes a body 110 having an entrance and an exit. The entrance and the exit are each configured to receive a tubing line 20 therethrough. Applying a force to the tubing line 20 causes the body 110 to restrict fluid flow through the tubing 20 line, thereby indicating a dislodgement condition, as already described above.

In an example, the body 110 has a first plate 112 and a second plate 114. The first plate 112 is connected by a hinge 116 to the second plate 114. A restriction tab 118 is formed on the first plate 112 (although it may be formed on the second plate 114 or both plates 112 and 114).

The tubing line 20 may be threaded through an entrance (e.g., formed through the hinge 116) into a first opening 120a in the first plate 112. The tubing line 20 is further threaded through a second opening 120b in the first plate 112, and through the exit (e.g., formed as an opening 120c in the second plate 114).

Applying the force to the tubing line 20 (e.g., in the direction of arrow 122a and/or arrow 122b) closes the first plate 112 against the second plate 114. This closure causes the restriction tab 118 to pinch the tubing line 20 between the first plate 112 and the second plate 114, as shown in FIG. 6B and thereby partially or fully restrict flow through the tubing line 20.

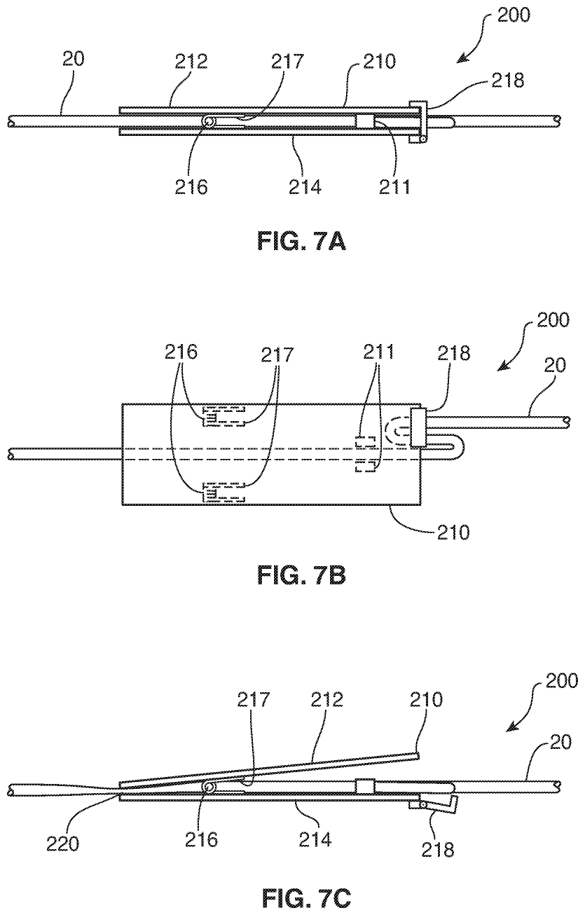

FIGS. 7A-7C show another example patient line dislodgement detection device 200. The example patient line dislodgement detection device 200 includes a body 210 having an entrance and an exit. The entrance and the exit are each configured to receive a tubing line 20 therethrough. A guide and/or spacer 2 is provided to support the tubing line 20. Applying a force to the tubing line 20 causes the body 210 to restrict fluid flow through the tubing line, thereby indicating a dislodgement condition as already discussed above.

In an example, the body 210 has a first plate 212 and a second plate 214. The first plate 212 is connected by one or more hinge 216 to the second plate 214. One or more spring member 217 biases the hinge(s) 216 in an open position. A trigger member 218 is hinged on either first plate 212 or the second plate 214 (as shown in FIG. 7A), and removably grasps the other plate to hold the first plate 212 generally parallel to the second plate 214 against a bias of the spring member 217.

Applying a force to the tubing line 20 pulls against and releases the trigger member 218. Releasing the trigger member 218 causes the first plate 212 and the second plate 214 to close under bias of the spring member 217. This action pinches the tubing line 20, e.g., at a vertex 220 formed between the first plate 212 and the second plate 214.

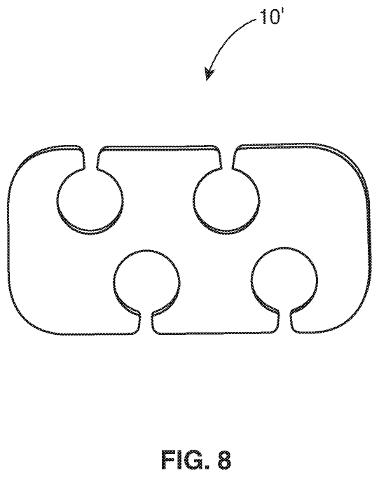

FIG. 8 shows another example patient line dislodgement detection device 10′. In this example, the device 10′ includes a body having slotted openings (corresponding to openings 12a-d of device 10). The openings are configured to receive a tubing line (not shown) for a patient and operates similar to the device 10 already described above. The slotted openings enable insertion of a tubing line without having to thread the tubing line through the holes (e.g., the tubing line can be inserted through the slots. That is, the device 10′ can be attached to the needle line after the needle line is manufactured (for example, at the clinic), and the “keyhole” slots can provide easy attachment but will still inhibit removal of the tube from the device thereby enabling it to operate as described above for device 10.

It is noted that the examples shown and described are provided for purposes of illustration and are not intended to be limiting. Still other examples are also contemplated.

1. A patient line dislodgement detection device, comprising a body for receiving a tubing line, the body restricting fluid flow by the dislodgement device through the tubing line in response to application of a force on the tubing line, the restricted fluid flow through the tubing line indicating a dislodgement condition.

2. The device of claim 1, further comprising the body restricting fluid flow in a needle line connected to a needle, catheter, or patient fluid connection.

3. The device of claim 1, further comprising the body restricting fluid flow in the tubing line to cause a physical change.

4. The device of claim 3, wherein the physical change is detected by at least one of pressure sensing, flow rate sensing, and other restriction sensing caused by a strain on the tubing line.

5. The device of claim 3, wherein the physical change is at least one of the following: geometric, optical, capacitive, resistive.

6. The device of claim 3, wherein the physical change is in a component of the dislodgement device.

7. The device of claim 3, wherein the physical change is in the tubing line.

8. The device of claim 3, wherein the physical change in a fluid in the tubing line.

9. The device of claim 1, wherein the force at least partially restricts fluid flow in the tubing line.

10. The device of claim 1, wherein the force directly occludes the tubing line to completely stop fluid flow through the tubing line.

11. The device of claim 1, further comprising a plurality of openings in the body, the tubing line threaded through the plurality of openings.

12. The device of claim 11, wherein a first and a second of the plurality of openings are both substantially D-shaped, and wherein the straight edge of the first and second openings are aligned substantially parallel to one another to provide a flat surface to occlude the tubing line when the force is applied to the tubing line.

13. A patient line dislodgement detection device, comprising:
a body having a plurality of openings formed therein configured to receive a tubing line therethrough; and
wherein applying a force to a tubing line causes the body to restrict fluid flow through the tubing line, thereby indicating a dislodgement condition.

14. The device of claim 13, wherein the body is generally rectangular shaped, and has a first opening, a second opening, a third opening, and a fourth opening, wherein the first opening and the third opening are formed in the body along a first threading line, and the second opening and the fourth opening are formed in the body along a second threading line, the first threading line spaced apart from the second threading line, and the first threading line substantially parallel to the second threading line.

15. The device of claim 14, wherein the tubing line is threaded over a first side of the body and through the first opening to a second side of the body, the tubing line is further threaded from the second side of the body through the third opening to the second side of the body, the tubing line is further threaded from the first side of the body through the fourth opening to the second side of the body, the tubing line is further threaded from the second side of the body through the second opening to the first side of the body.

16. The device of claim 14, wherein first opening is offset from the fourth opening, and wherein the second opening is offset from the third opening.

17. The device of claim 13, wherein a first and second of the plurality of openings each have a straight edge aligned substantially parallel to one another to provide a flat surface to occlude the tubing line when the force is applied to the tubing line

18. The device of claim 13, wherein a first and a second of the plurality of openings are both substantially D-shaped.

19. The device of claim 18, wherein a straight edge of the first and second D-shaped openings are aligned substantially parallel to one another.

20. The device of claim 19, wherein the straight edge of the first and second D-shaped openings to provide a flat surface to occlude the tubing line when the force is applied to the tubing line.
